(19) United States
(12) Patent Application Publication
(10) Pub. No.: US 2004/0009584 A1
(43) Pub. Date: Jan. 15, 2004

(54) Method for manufacturing microarrays based on the immobilization of porous substrates on thermally modifiable surfaces

(76) Inventors: Rahul Mitra, Pearland, TX (US); Michael E. Hogan, Tuscon, AZ (US)

Correspondence Address: FULBRIGHT & JAWORSKI, LLP, 1301 MCKINNEY, SUITE 5100, HOUSTON, TX, 77010-3095, US

(21) Appl. No.: 10/388,061

(22) Filed: Mar. 13, 2003

Related U.S. Application Data
(60) Provisional application No. 60/364,155, filed on Mar. 13, 2002.

(51) Int. Cl. C12M 1/34; B05B 1/00; B32B 31/00

(52) U.S. Cl. 435/287.2; 156/280

(57) ABSTRACT

The present invention is directed to a method of manufacturing a high density analysis device, preferably a microarray, comprising attaching an activatable material to a substrate, such that the activatable material and the substrate are in direct contact, to form a surface material; ii) immobilizing a porous or non-porous material onto the surface material; and iii) binding the immobilized porous or non-porous material with a probe to provide the high-density analysis device, and articles of same.

### CROSS REFERENCE TO RELATED APPLICATIONS

This application claims priority to U.S. provisional application serial number 60/364,155, filed Mar. 13, 2002.

### FIELD OF THE INVENTION

The present invention provides a method of manufacturing a high density analysis device by attaching an activatable material to a substrate, such that the activatable material is in direct contact with the substrate; immobilizing a porous or non-porous material onto the surface material by a physical means; and reacting an inorganic molecule, an organic molecule, a bio-molecule, a cell, or a organelle onto the porous or non-porous material to provide an element, wherein the device comprises a plurality of elements, wherein each element has a characteristic probe. The article of manufacture is also provided and is useful as a microarray, a bio-sensor or a chemosensor.

### BACKGROUND OF THE INVENTION

A rapid explosion in the sequencing of entire genomes drives the need for highly parallel methods that allow simultaneous investigation of several thousands of genes in a highly miniaturized fashion. Parallel study of thousands of genes at the genomic level promises to be a critical element in understanding and curing disease. For this reason, among others, high-throughput analysis methods are imperative to the future of pharmaceuticals including gene discovery, disease diagnosis, genotyping, protein expression, elucidating metabolic responses, drug design, drug discovery and toxicology.

One such method to investigate several thousands of molecules in parallel is an array (Shi, 2002). Briefly, an array is an orderly arrangement of samples and serves as a medium for matching samples based on complementarity. One specific array is a microarray, which is distinguished by samples sizes of less than 200 microns in diameter. The microarray is, in one sense, a bio-sensor device comprising a probe, which is a biomolecule of known identity, attached to a surface coating of a substrate or solid support. The probe is applied iteratively to the substrate in a highly parallel fashion to generate a discrete spatial grid such that an array having elements corresponding to a particular probe is produced. A target is a molecule to be analyzed which is typically of unknown identity and in some cases is extracted from a sample of interest and labeled with a fluorescent dye. The labeled target(s) are incubated with the microarray under hybridizing conditions and allowed to bind to its complementary probe on the array. After removing the unbound target, the amount of bound target is detected in a specific pattern and quantitated.

Two main ways of preparing a microarray using flat plain glass as substrates have been described- light directed in-situ synthesis of a probe, and immobilization of synthesized biomolecules onto solid substrates that serve as probes for the microarray (WO 90/03382; WO 93/22680; U.S. Pat. No. 5,412,087 to McGall et al.; WO 95/15970).

In addition, several other methods are emerging that use unconventional means, namely, microfluidics (for example, U.S. Pat. No. 5,900,215) and beads immobilized on electrodes and chemically etched ends of optical fibers. The ease and quality of manufacturing these microarrays are limited by the methodology employed. For example, in-situ synthesis requires special instrumentation that is not common laboratory equipment and quality control is difficult. Similarly, immobilization of synthesized biomolecules (probes) onto a coated glass substrate suffers from high background, low surface area and low probe density.

To improve the latter, WO 00/61282 to Affymetrix, Inc. teaches porous substrates prepared from primarily inorganic compounds (e.g., silica-containing compounds) on which polymers are bound. Genetic diagnostic devices comprising the porous substrate bound to a polymer that offer a high polymer density for a two dimensional area without changing the spacing between polymers on the surface of the porous substrate are also taught. However, WO 00/61282 teaches a pore size of 1-500 nm and a porous surface thickness of 0.01 to 20 mm, which limits the maximum amount of target that is analyzed on one device.

A different approach to improve low probe density is described by Nagasawa et al. (U.S. published application 2001/0039072) which teaches reactive probe chip comprising a composite substrate having porous micro-compartments (i.e., wells) within which loaded porous carrier particulate probes are immobilized. Nagasawa et al. teaches that it is critical that the immobilization of the carrier particulate probes occur only at the outer surfaces and protective measures such as impregnating with water are taught to prevent damage to the inner pore surface, which carries the bound probe, during immobilization.

However, not all methods that employ beads as substrates to immobilize biomolecules suffer from the same issues. A major problem with bead arrays is anchoring the bead to a support in a manner that spatially separates one type of bead from the other. One attempt described to mitigate this problem is trapping a specific dye inside the bead to produce a bead having an individual spectral address and, thus, more readily detected. The amount of bound target is estimated by fluorescence of the dye with which the targets are labeled. A limiting factor of this method is the number of spectral addresses that are available and the time-intensity of sorting, quantitating, and aggregating the beads. As noted, a further problem with bead arrays is sorting, and employing traditional means such as flow cytometric devices, wherein each bead is sorted as it individually passes through a gate of defined dimension, must be used to overcome this issue. Currently, a set of 100 beads with individual spectral addresses is commercially available (Luminex Corporation).

In order to overcome the problems associated with the free floating beads such as clumping and aggregation, a new system using fiber optics has been described by Walt et al. and disclosed the characterization of 100,000 spectral addresses (U.S. Pat. No. 6,327,410). A substrate, preferably an optical fiber, is etched to form a cavity which provides a means to immobilize a bead. One optical fiber carries solely one bead, and, thus, the arrays need not be ordered. This method offers several advantages over the free solution bead sorting method, but hints at being expensive and difficult to make.

One method for making ordered arrays of DNA on a porous membrane is a “dot blot” approach. In this method, a vacuum manifold transfers a plurality, e.g., 96, aqueous samples of DNA from 3 millimeter diameter wells to a porous membrane. A common variant of this procedure is a “slot-blot” method in which the wells have highlyelongated oval shapes, and the DNA is immobilized on the porous membrane by baking the membrane or exposing it to UV radiation. This is a manual procedure that is practical for preparing one array at a time and is usually limited to 96 samples per array. “Dot-blot” procedures are therefore inadequate for applications in which many thousand samples must be analyzed.

A more efficient technique employed for making ordered arrays of genomic fragments uses an array of pins dipped into the wells, e.g., the 96 wells of a microtitre plate, for transferring an array of samples to a substrate, such as a porous membrane. One array includes pins that are designed to spot a membrane in a staggered fashion, for creating an array of 9216 spots in a 22×22 cm area (Lehrach, et al., 1990). A limitation with this approach is that the volume of DNA spotted in each pixel of each array is highly variable. In addition, the number of arrays that are made with each dipping is usually quite small, thereby leading to long manufacturing times.

An alternate method of preparing ordered arrays of nucleic acid sequences is described by Pirrung, et al. (1992) and by Fodor, et al. (1991). The method involves synthesizing different nucleic acid sequences at various discrete regions of a support. This method employs elaborate synthetic schemes and is generally limited to relatively short nucleic acid samples, e.g., less than 20 bases. A related method has been described by Southern, et al. (1992).

Khrapko, et al. (1991) describes a method of making an oligonucleotide matrix by spotting DNA onto a thin layer of polyacrylamide. The spotting is done manually with a micropipette. These methods described in the prior art lack design for mass fabrication of microarrays characterized by (i) a large number of micro-sized assay regions separated by a distance of 50-200 microns or less, and (ii) a well-defined amount, typically in the picomole range, of analyte associated with each region of the array. Furthermore, current technology is directed at performing such assays one at a time to a single array of DNA molecules. For example, a most common method for performing DNA hybridizations to arrays spotted onto porous membrane involves sealing the membrane in a plastic bag (Maniatas, et al., 1989-MOLECULAR CLONING, A LABORATORY MANUAL, Cold Spring Harbor Press (1989)) or a rotating glass cylinder (Robbins Scientific) with the labeled hybridization probe inside the sealed chamber.

U.S. Pat. No. 5,807,522 to Brown et al. teaches a spotting method of fabricating microarrays for biological samples in which a solid support having a discrete sample-analysis region prepared by applying a selected, analyte-specific reagent to the solid support using an elongate capillary channel and a tip region at which the solution in the channel forms a meniscus, tapping the tip of the dispensing device against the solid support at a defined position on the surface, with an impulse effective to break the meniscus in the capillary channel and depositing a selected volume between 0.002 and 2 nl of solution on the surface. Iterative steps of depositing the analyte-specific reagent to the solid support produces the final microarray. Brown et al. also teaches that the solid support comprises a substrate having a water impermeable backing, and atop the backing is a water permeable film formed of a porous or a non-porous material at a thickness of between 10 to 1000 microns. A grid in formed on the solid support by applying a barrier material, such as silicon, by mechanical pressure or printing to form a water-proof barrier separating regions of the solid support.

For arrays made on non-porous surfaces, such as a microscope slide, each array is incubated with the labeled hybridization probe sealed under a coverslip. These techniques require a separate sealed chamber for each array which makes the screening and handling of many such arrays inconvenient and time intensive.

Methods that optimize other parameters such as immobilization of a probe to a substrate have been described. U.S. Pat. No. 5,900,481 to Lough et al. and related patent U.S. Pat. No. 6,133,436 to Koster et al. teach immobilization of a nucleic acid via conjugation to a bead that is further conjugated through a covalent or ionic attachment to a solid support. U.S. Pat. No. 5,837,196 to Pinkel et al. teaches a biosensor comprising a plurality of optical fibers having biological binding partners attached to the sensor end, thereby providing a high density sensor for biomolecules. U.S. Pat. No. 5,436,327 to Southern et al. teaches synthesizing oligonucleotides by solid-phase methodology, wherein the linkage is a non-labile phosphodiester which is further linked to a hydrophilic spacer to affix to the solid support.

U.S. Pat. No. 6,139,831 to Shivashankar et al. describes a method of immobilizing a biomolecule onto a substrate using a specific film having a low conductivity and a low melting temperature, namely a gold film. An applied electromagnetic radiation melts and ablates the film at the impingement site. The film is in contact with a colloidal dispersion and upon melting generates a convective flow at the reaction site, thereby leading to adhering of an insoluble particle in the dispersion to the specifically melted site. Shivashandar et al. teach that the insoluble particle is from 10 nanometers to a few micron in diameter and is conjugated with the biomolecule of interest. The success of this manufacturing method relies on the film absorbing energy of the beam primarily at the impingement site so that local melting and ablation of the film occur.

The present invention fulfills a long-sought need in the art by providing an effective means of manufacturing spatially addressable three-dimensional microarrays comprised of coated porous materials immobilized on a surface without spotting, thereby allowing for increased uniformity and reproducibility. Because the present invention immobilizes a porous or non-porous material by embedding in an activatable material, the resulting three-dimensional microstructure provides the advantages associated with flat surfaces, namely,, large surface area, higher probe density, individual addressability of each element, and higher density of different probes/array, low background, and ease of use. The inventive analysis device is reusable. The methods of manufacturing the inventive high density analysis device are time-efficient and cost effective. Further, the high-density analysis device provides high probe density and versatile analytical utility by using a porous or non-porous material that, after immobilization, substantially provides an increased surface area and three-dimensional structure for analysis.

### SUMMARY OF THE INVENTION

In the present invention, there is a method of manufacturing a high-density molecular analysis device comprising i) attaching an activatable material to a substrate, such that the activatable material and the substrate are in direct contact, to form a surface material; ii) immobilizing a porous material onto the surface material; iii) reacting the porous material with a probe to generate an element, and iv) repeating step iii) to provide the high-density molecular analysis device comprising a plurality of elements, wherein each element is homogeneous for the probe of that element.

In one embodiment of the method, the step of reacting the porous material with the probe is performed before the step of immobilizing the porous material. In another embodiment of the method, the activatable material is characterized by having an activating temperature of less than 150° C.

In a specific embodiment, the activatable material is a polymer. In a further specific embodiment, the polymer is agarose. In a specific embodiment wherein the polymer is agarose, the probe is a cell.

In another embodiment of the method, the activatable material is a sol-gel glass.

In another embodiment of the method, the activatable material comprises an adhesive, wherein the adhesive is thermally-cured. In a specific embodiment wherein the adhesive is thermally-cured, the adhesive is an epoxide, an acrylic, a wax, a resin or an organic molecule that functions as a synthetic support.

In another embodiment of the method, the activatable material comprises an adhesive, wherein the adhesive is photo-cured. In a specific embodiment wherein the adhesive is photo-cured, the adhesive is an epoxide or an acrylic.

In another embodiment of the method, the step of immobilizing is achieved by trapping the porous material onto the surface material, wherein the surface material is in direct contact with at least one pore of the porous material.

In another embodiment of the method, the porous material is characterized by having a diameter of at least about 1 micrometer.

In another embodiment of the method, the porous material comprises a synthetic polymer.

In another embodiment of the method, the porous material comprises a biomolecular aggregate.

Also in the present invention, there is a method of manufacturing a high-density molecular analysis device comprising i) attaching an activatable material to a substrate, such that the activatable material and the substrate are in direct contact, to form a surface material; ii) immobilizing a porous or non-porous material onto the surface material; iii) reacting the porous or non-porous material with a probe to generate an element, and iv) repeating step iii) to provide the high-density molecular analysis device comprising a plurality of elements, wherein each element is homogeneous for the probe of that element.

In the present invention, there is also method of detecting a bio-molecule comprising the steps of i) attaching an activatable material to a substrate, such that the activatable material and the substrate are in direct contact, to form a surface material, ii) immobilizing a porous material onto the surface material, iii) reacting the porous material with a probe to provide an element, iv) repeating step iii) to provide a high-density analysis device comprising a plurality of elements having a characteristic probe, v) applying a sample comprising a bio-molecule to the high-density analysis device, vi) binding the bio-molecule to the probe, and v) detecting the binding.

In one embodiment of the method, the step of reacting the porous material with the probe is performed before the step of immobilizing the porous material.

In another embodiment of the method, the activatable material is characterized by having an activating temperature of less than 150° C.

T In another embodiment of the method, the activatable material comprises an adhesive, wherein the adhesive is thermally-cured.

In another embodiment of the method, the step of immobilizing is achieved by trapping the porous material onto the surface material, wherein the surface material is in direct contact with at least one pore of the porous material.

In another embodiment of the method, the porous material comprises an organism having a dimension in the range of about 1 micrometer to about 100 millimeters.

In another embodiment of the method, the porous material is characterized by having a diameter of at least about 1 micrometer.

In another embodiment of the method, the probe comprises a nucleic acid sequence, wherein the nucleic acid sequence is complementary to a nucleic acid sequence of the bio-molecule.

In another embodiment of the method, the probe comprises a polypeptide having a complementary molecule, wherein the bio-molecule comprises the complementary molecule.

In another embodiment of the method, the probe is an antibody having a complementary antigen, wherein the bio-molecule is the antigen.

In another embodiment of the method, the probe is a polypeptide having a biological activity, wherein the bio-molecule alters the biological activity of the polypeptide.

In another embodiment of the method, the probe is labeled with a compound that fluoresces. In a specific embodiment of the method using a compound that fluoresces, the step of detecting comprises fluorescence spectroscopy.

In another embodiment of the method, the probe is a molecule that alters a biological activity of a polypeptide, wherein the bio-molecule comprises the polypeptide.

Also in the present invention, there is also method of detecting a biomolecule comprising the steps of i) attaching an activatable material to a substrate, such that the activatable material and the substrate are in direct contact, to form a surface material, ii) immobilizing a porous or non-porous material onto the surface material, iii) reacting the porous or non-porous material with a probe to provide an element, iv) repeating step iii) to provide a high-density analysis device comprising a plurality of elements having a characteristic probe, v) applying a sample comprising a bio-molecule to the high-density analysis device, vi) binding the bio-molecule to the probe, and v) detecting the binding.

In the present invention, there is also a method of manufacturing a high-density bio-reactor comprising the steps of i) attaching an activatable material to a substrate, such that the activatable material and the substrate are in direct contact, to form a surface material, ii) immobilizing a porous material onto the surface material, iii) reacting the porous material with a probe to generate an element; iv) repeating step iii) to provide the high-density bio-reactor comprising a plurality of elements having a characteristic probe, v) applying a sample comprising a target to the high-density bioreactor, wherein an interaction between the target and the probe produces a detectable product; and vi) detecting the product.

In one embodiment of the method, the step of reacting the porous material with the probe is performed before the step of immobilizing the porous material.

In another embodiment of the method, the activatable material is characterized by having an activating temperature of less than 150° C.

In another embodiment of the method, the activatable material comprises an adhesive, wherein the adhesive is thermally-cured.

In another embodiment of the method, the step of immobilizing is achieved by trapping the porous material onto the surface material, wherein the surface material is in direct contact with at least one pore of the porous material.

In another embodiment of the method, the target is a polypeptide of an amino acid sequence of an enzyme. In a specific embodiment wherein the target is a polypeptide, the probe is an organic molecule that serves as a substrate for said enzyme.

In the present invention, there is also a method of manufacturing a high-density bio-reactor comprising the steps of i) attaching an activatable material to a substrate, such that the activatable material and the substrate are in direct contact, to form a surface material, ii) immobilizing a porous or non-porous material onto the surface material, iii) reacting the porous or non-porous material with a probe to generate an element; iv) repeating step iii) to provide the high-density bio-reactor comprising a plurality of elements having a characteristic probe, v) applying a sample comprising a target to the high-density bio-reactor, wherein an interaction between the target and the probe produces a detectable product; and vi) detecting the product.

In yet another embodiment of the present invention, there is a method of detecting expression of a polypeptide in a sample comprising the steps of i) attaching an activatable material to a substrate, such that the activatable material and the substrate are in direct contact, to form a surface material, ii) immobilizing a porous material onto the surface material, iii) reacting a unit of the porous material with a probe, wherein the unit is homogenous for the probe, iv) repeating step iii) to provide to a high density bio-sensor comprising a plurality of units, wherein each unit is homogeneous for a probe, v) applying the sample comprising the polypeptide to the high-density bio-sensor; vi) binding the polypeptide to the probe of at least one unit, and vii) detecting the binding, wherein the detection indicates expression of the polypeptide in the sample.

In one embodiment of the method, the step of reacting the porous material with the probe is performed before the step of immobilizing the porous material.

In another embodiment of the method, the activatable material is characterized by having an activating temperature of less than 150° C.

In another embodiment of the method, the activatable material comprises an adhesive, wherein the adhesive is thermally-cured.

In another embodiment of the method, the step of immobilizing is achieved by trapping the porous material onto the surface material, wherein the surface material is in direct contact with at least one pore of the porous material.

In another embodiment of the method, the probe comprises a complementary DNA, an oligonucleotide, a chromosome, a PCR product or a gene fragment.

In another embodiment of the method, the probe comprises a polypeptide.

In another embodiment of the method, the probe comprises an antibody.

In other embodiments, either in the alternative or in combination with other embodiments, the probe could be a synthetic small molecule, a whole viral molecule, a viral coat protein assembly, a cell, a whole viral particle, and/or a microorganism.

Also in the present invention, there is a method of detecting expression of a polypeptide in a sample comprising the steps of i) attaching an activatable material to a substrate, such that the activatable material and the substrate are in direct contact, to form a surface material, ii) immobilizing a porous or non-porous material onto the surface material, iii) reacting a unit of the porous or non-porous material with a probe, wherein the unit is homogenous for the probe, iv) repeating step iii) to provide to a high density bio-sensor comprising a plurality of units, wherein each unit is homogeneous for a probe, v) applying the sample comprising the polypeptide to the high-density bio-sensor; vi) binding the polypeptide to the probe of at least one unit, and vii) detecting the binding, wherein the detection indicates expression of the polypeptide in the sample.

There is also a method of manufacturing a high-density molecular analysis device comprising attaching a molecule or cell to a substrate, such that the molecule or cell and the substrate are in direct contact, to form a surface material, immobilizing a porous material onto the surface material by thermal modification, reacting the porous material with a probe to generate an element; and repeating the step of reacting the porous material to provide the high-density molecular analysis device comprising a plurality of elements, wherein each element is homogeneous for the probe of that element.

There is also a method of manufacturing a high-density molecular analysis device comprising attaching a molecule or cell to a substrate, such that the molecule or cell and the substrate are in direct contact, to form a surface material, immobilizing a porous or non-porous material onto the surface material by thermal modification, reacting the porous or non-porous material with a probe to generate an element; and repeating the step of reacting the porous material to provide the high-density molecular analysis device comprising a plurality of elements, wherein each element is homogeneous for the probe of that element.

Other and further objects, features, and advantages are apparent and eventually more readily understood by reading the following specification and the accompanying drawings forming a part thereof, or any examples of the presently preferred embodiments of the invention given for the purpose of the disclosure.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1 illustrates the coating of a substrate with a thermally activatable surface.

FIG. 2 illustrates the coating of a substrate with a sol-gel activatable surface by a chemical means.

FIG. 3 illustrates immobilization of the porous or non-porous material on the surface material.

FIGS. 4A and 4B depict a cross-section of a porous or non-porous sphere A) before immobilization, and B) after immobilization to the surface material.

FIG. 5A shows beads before imbedding into the paraffin surface. FIG. 5B shows beads imbedded into the paraffin surface by thermal activation.

FIG. 6A shows the specificity, as seen by fluorescence intensities, obtainable with the present method. FIG. 6B shows the results in graphical form.

### DESCRIPTION OF THE INVENTION

It will be readily apparent to one skilled in the art that various embodiments and modifications may be made in the invention disclosed herein without departing from the scope and spirit of the invention.

As used in the specification, “a” or “an” may mean one or more. As used in the claim(s), when used in conjunction with the word “comprising”, the words “a” or “an” may mean one or more than one. As used herein “another” may mean at least a second or more.

The technology of the present invention is related to the invention described in the U.S. Patent Application entitled, “[TITLE OF DEVICE APPLICATION]” filed on the same day and incorporated by reference herein.

By “array” herein is meant a plurality of bioactive agents in an array format; the size of the array will depend on the device and end use of the array. Arrays containing from about 2 different bioactive agents, wherein a bioactive agent is a porous or non-porous material coated with a probe, to many millions are made by the methods described herein. Generally, the array comprises from two to as many as a billion or more, depending on the size of the porous or non-porous material, such as a bead, and the substrate, as well as the end use of the array, thus very high density, high density, moderate density, low density and very low density arrays are made. Preferred ranges for very high density arrays are from about 10,000,000 to about 2,000,000,000, with from about 100,000,000 to about 1,000,000,000 being preferred. High density arrays range about 100,000 to about 10,000,000, with from about 1,000,000 to about 5,000,000 being particularly preferred. Moderate density arrays range from about 10,000 to about 50,000 being particularly preferred, and from about 20,000 to about 30,000 being especially preferred. Low density arrays are generally less than 10,000, with from about 1,000 to about 5,000 being preferred. Very low density arrays are less than 1,000, with from about 10 to about 1000 being preferred, and from about 100 to about 500 being particularly preferred. In specific embodiments, the device of the present invention is not be in array format; that is, for some embodiments, devices comprising a single bioactive agent are made as well. Additionally, arrays having multiple substrates are contemplated, either of different or identical compositions. Thus for example, large arrays comprise a plurality of smaller substrates.

The term “activatable surface” refers to a surface that has the ability to alter by interaction with a physical, chemical or an artificial energy. Such energy includes Xrays, cathode rays, cosmic rays, planetary rays, electromagnetic radiation, primary and secondary radiation from radioactivity, chemluminescence, bioradiation, osmosis, dialysis, electrochemical gradients, electricity, magnetism, chemicals, force, shear forces, and pressure. Surfaces that are “activatable” include glass, acrylics, epoxides, waxes, resins, natural and synthetic polymers; organic molecules that function as synthetic supports, heat sealing papers, thermally-cured adhesives or materials, photo-cured adhesives, and adhesives. By “alter” as used with respect to the activatable material is meant that the activatable material undergoes a transition in a physical state, such as melting and in that event is reversible, or a transition in a chemical state. In a specific embodiment, the activatable material is melted and applied to a surface of the substrate to form a surface material. In another specific embodiment, the activatable material is mixed with a reagent, such as an organic solvent to effect dissolution or an activator to effect activation, and applied to a surface of the substrate to form a surface material. A non-limiting example of a chemical alteration occurs on applying an electromagnetic radiation to a polymer to generate free radicals, thereby providing a means to immobilize a probe to the porous or non-porous material (i.e., by crosslinking). In this case, the crosslinking is considered irreversible.

The term “biosensor” as used herein refers to a sensor that detects chemical species with high selectivity on the basis of molecular recognition rather than the physical properties of analytes. See, e.g., Advances in Biosensors, A. P. F. Turner, Ed. JAI Press, London, (1991). Many types of biosensing devices have been described, including enzyme electrodes, optical immunosensors, ligand-receptor amperometers, and evanescent-wave probes. Updike and Hicks, Nature, 214: 986 (1967), Abdel-Latif et al., Anal. Lett., 21: 943 (111988); Giaever, J. Immunol., 110: 1424 (1973); Sugao et al. Anal. Chem., 65: 363 (1993), Rogers et al. Anal. Biochem., 182: 353(1989).

The term “porous material” refers to a material comprising a pore in its surface. The pore is continuous throughout its body or extends to a depth that is less than the depth of the material's body. The size of a pore is a fraction of the body of the material and varies with a porous material. The pore size need not be controlled provided that the porous material is a diameter of at least 1 μm. The shape of the porous material is a geometric shape such as a sphere, a cube, a pyramid, or other three dimensional shapes known to one of ordinary skill in the art. Further, the shape of the pore need not be a defined geometrical shape provided it is three dimensional or two dimensional, such as a bead, a rod, a fiber and a tile. Specific embodiments contemplated include, but are not limited to, glass, inorganic elements, metals, inorganic compounds such as fluorinated compounds, plastics, carbons such as buckminsterfullerenes and derivatives thereof, cotton fibers, natural and synthetic polymers, bio-molecular aggregates, such as gelatin, alginate, protein, DNA films, RNA, carbohydrate-crosslinked gels, polysaccharides, collagen, fiber, keratin, or a bio-molecule that aggregates spontaneously or by a non-self means to generate a shape having pores such as micelle formation of phospholipids), synthetic aggregates (i.e., synthetic glass, biotin, dextran, a crosslinkable material such as polyethylene, nylon, Dacron, paper), a cell, tissue, an organelle, and organisms having dimensions between about 0.001 nm to about 100 mm.

The term “probe” refers to a molecule, a nucleic acid, a polypeptide, an antibody or a compound of natural or synthetic origin. Chemical synthesis of polypeptides is known in the art and are described further in Merrifield, J., J. Am. Chem. Soc., 91:501(1969); Chaiken, I. M., CRC Crit. Rev. Biochem., 11:255 (1981); Kaiser et al., Science, 243:187 91989); Merrifield, B., Science, 232:342 (1986); Kent, Ann. Rev. Biochem., 57:957 (1988); and Offord, R. E., Semisynthetic Proteins, Wiley Publishing (1980). In addition, methods for chemical synthesis of peptide, polycarbamate and polynucleotide arrays have been reported (see Foder et al., Science, 251:767-773 (1991); Cho et al., Science, 261:1303-1305 (1993)). In the present invention, the probe is coated, bound, reacted, or adhered to a porous material. Functionally, the probe is complementary to a molecule, a nucleic acid, a polypeptide or compound of natural or synthetic origin that serves as a target, wherein the target is in a sample that is to be analyzed. In a specific embodiment, a probe is a tethered nucleic acid with known sequence and the target is a free nucleic acid sample whose identity and/or abundance is detected by complementary binding of the probe. In another specific embodiment, the probe is a polypeptide with a known or unknown amino acid sequence having a known biological activity and the target is an organic molecule, wherein after binding of the probe to the target, the biological activity is detected either by a decrease in the biological activity or an increase in the biological activity as compared to the native biological activity of the polypeptide. Non-limiting examples of a probe useful in the present invention includes inorganic compounds such as inorganic metals or salts; organic molecules such as dyes, drugs, amino acids, small ligands, and synthetic organic compounds; bio-molecules such as DNA, RNA, PNA (protein nucleic acid), a protein, carbohydrate, amino acids, antibodies, cells, and organelles. A skilled artisan recognizes that bio-molecules are also correctly considered natural polymers, for example, DNA and RNA and proteins are natural polymers.

The term “polymer” as used herein refers to a natural or a synthetic polymer unless otherwise noted. Synthetic polymers that are useful in the present invention include polyhydrocarbons, nylon, polyesters and polycarbonates and are in a form of a powder, resin particles or a preform such as a consolidated bar, block, rod or any other shape. In a preferred embodiment, the polymer is in the form of a powder or resin particles if a thermal deposition of the porous or non-porous material is desired to immobilize the porous or non-porous material on the surface material. A non-limiting example of a natural polymer is agarose.

The terms “polypeptide” and “protein” as used herein are interchangable and refer to a gene product encoded by a nucleic acid sequence.

A “sample” as used herein refers to a molecule, a protein, a compound, an extract, a solution, a slurry, an emulsion, a colloidal dispersion, a cell, and/or organelle that is of interest to the user and comprises a target. For example in a specific embodiment, the sample is a nucleic acid obtained from a cell of an organism in a living or dead state, from an artificial cell culture or from a natural source in a fresh, boiled or frozen state. Methods of obtaining a nucleic acid from a cell are well known in the art.

The analytical devices of the present invention comprise a substrate. By “substrate” or “solid support” or other grammatical equivalents as used herein is meant any material that is modified by applying an activatable material which is appropriate for the attachment or association of a porous material and is amenable to at least one detection method. As appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, Teflon™, and the like), polysaccharides, nylon or nitrocellulose, resins, silica or silica-based materials including silicon and modified silicon, carbon, metals, inorganic glasses, plastics, and a variety of other polymers. In a specific embodiment, a binding of a target to a probe is detected by fluorescence, and in this case, one of ordinary skill in the are recognizes that the substrate allows optical detection and does not appreciably fluoresce. Similar considerations of detection methods are contemplated prior to manufacturing the high-density analysis device to allow for the lowest limit of detection and highest signal-to-noise ratio in the system.

Generally the substrate is planar, although it is appreciated by those in the art, other configurations of substrates are contemplated as well; for example, three dimensional configurations are used. Preferred substrates include glass, polystyrene and other plastics and acrylics.

The immobilization of porous or non-porous surfaces of the present invention employs the principle that activatable materials such as glues, adhesives, resins, and waxes undergo a transition from one physical state to another state. This is similar to the melting of the candle wax while hot and solidifying upon cooling. The underlying principle is to use an activatable material that after activating effects a physical or chemical alteration in the material (i.e., melting or softening of a solid to a near-liquid). A porous or non-porous material is applied to the activated material in a spatially addressable manner to generate an array of the porous or non-porous material. In one embodiment, the porous or non-porous material immobilized on the surface material is then coated with a probe of interest which is used as a sensor to identify, bind, and/or quantitate a complementary target.

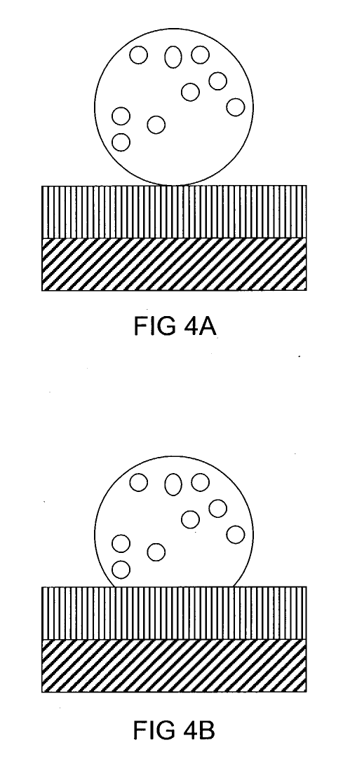

For example, the surface material is heated by applying a thermal energy to a temperature that is above ambient temperature, 25° C., but below 150° C., thereby reversibly activating by heating for a time sufficient to effect softening of (i.e., activate) the surface material. The absorption of energy occurs throughout the material. The porous or non-porous material comprises pores within which the surface material permeates, invades or infuses the porous or non-porous material to trap, or otherwise, immobilize the porous or non-porous material onto the surface material. A skilled artisan recognizes that the step of immobilization occurs in the liquid-solid state (softened solid) or the solid-state such as a wax that is in the solid state. Further a chemical reaction is not taking place between the porous or non-porous material and the surface material (see, FIGS. 4A and 4B). In a specific embodiment the surface material having the porous or non-porous material trapped, physically adhered to, and/or partially embedded within is allowed to cure, for example, by cooling at ambient conditions if the surface material is activated by heating. It is preferred that the porous material is not wholly in direct contact with the surface material, but rather, that at least one pore of the porous material is not in direct contact with the surface material.

The porous or non-porous material may be reacted, or charged, with a probe prior to immobilization onto the surface material. In this case, the probe is attached to the porous or non-porous material, wherein the attachment occurs both on the outer and inner surfaces of the material. The coated porous or non-porous material is then immobilized on the surface material by trapping the material onto the activated surface material through a direct contact therewith.

In, general, for a probe and complementary target to find each other in a binding reaction, the probe concentration must exceed the target concentration. If the probe concentration is lower that the target concentration, the dynamic range of probe-target interactions decreases.

For example, a typical solid bead of 5 μm diameter yields a surface area (4πr² ) of 78.5 μm². However, a solid bead of 0.1 μm diameter has a surface area of 0.03032 μm². Hence, the surface area of a 5 μm solid bead is 2589 times higher than the that of a 0.1 μm solid bead. Adding pores to the bead, substantially increases the surface as follows: if a pore size is 10 nm and bores through a bead, the surface area of the bead now calculated by using the following equation:

[(Area of pore)×(number of pores)]−[area of the bead lost to the pore]

By way of example, consider r, the radius of the pore, to be 0.25 microns, L is the length of the pore, and Y is the radius of the cylinder is 2.5 microns. The surface area of the cylinder (2πrL+2πr²) is first calculated. The number of pores that are formed is equal to the area of bead divided by the area of pore. In this example, the number of pores for a bead of the given dimensions is then 78.5/0.19625=400 pores. The total surface area of the bead with 400 pores is calculated as 400 {[(Area of pore)×(number of pores)]−[area of the bead lost to the pore], or 400×{(9.42−1.57)}, or 3140μm². Therefore, the area of a porous 2.5 micron bead with pores is 3140 μm² and without pores is 78.5 μm². Compare to a solid sphere of having a 0.05 μm² radius, which has a surface area of 0.03032 μm², the present invention provides a net improvement in surface area of 103,562 fold.

In one embodiment of the present invention is a method of manufacturing a high-density molecular analysis device comprising attaching an activatable material to a substrate, such that the activatable material and the substrate are in direct contact, to form a surface material, immobilizing a porous or non-porous material onto the surface material, reacting the porous or non-porous material with a probe to generate an element, and repeating the step of reacting the porous or non-porous material to provide the high-density molecular analysis device comprising a plurality of elements, wherein each element is homogeneous for the probe of that element.

In a specific embodiment, the step of reacting the porous or non-porous material with the probe is performed before the step of immobilizing the material. For example, a positively charged porous or non-porous material is reacted (i.e., mixed) with a nucleic acid having a negative charge to produce a porous or non-porous material coated with the nucleic acid probe that is attached by way of an ionic interactions. The coated porous or non-porous material is then immobilized onto the surface material, which is in a near-liquid or solid state. In the case of the former, the surface material invades the pores of the coated porous material, then hardens to immobilize the coated porous material. In the case of the latter, the coated porous material is applied to the surface material, and then the porous material is immobilized by heating the entire system to a temperature the softens the surface material or, for example the activatable material is a wax, applying a pressure atop the coated porous material to effect immobilization into the surface material by partially embedding the coated porous material into the surface material. It is preferred that in embodiments that employ heat to effect immobilization, the activatable material is characterized by having an activating temperature of less than 150° C.

In some cases, the activatable material is a polymer, such as agarose. In the case of the activatable material comprising a natural polymer such as agarose, the probe is, for example, a cell or an organelle or a tissue. The substrate is, in further specific embodiments, gel-bond paper which is commercially available.

In other specific applications, the activatable material is an artificial polymer such as a nanotube, nanoassembly of nanowires and the like, comprising of carbon, metals, or even nucleic acids.

In other variations, the activatable material is a sol-gel glass or an adhesive that is thermally-cured. By “adhesive” is meant a molecule or compound or polymer that is characterized by a sticky, gel-like, viscous, near-liquid physical state at the time of application and later hardens to a non-sticky, solid state. Examples of thermally-curable adhesives include, but are not limited to, epoxides, acrylics, a wax, a resin or an organic molecule that functions as a structural support such as a dendrimer. In another specific embodiment, the adhesive is photo-cured, and by way of examples includes epoxides, acrylics such as those commercially available from Dymax Corp. (see, Bachmann et al., “Advances in Light Curing Adhesives”). It is known in the art that UV adhesives cure at an exposure to electromagnetic radiation at a wavelength of 300-400 nm, and UV-Vis adhesives cure at a wavelength in the range of 300-500 nm.

By way of non-limiting example, the step of immobilizing may be achieved by trapping the porous material onto the surface material, wherein the surface material is in direct contact with at least one pore of the porous material.

In another specific embodiment, the porous or non-porous material is characterized by having a diameter of at least about 1 micrometer. One of ordinary skill in the art recognizes that the device of the present invention is a three-dimensional high-density molecular analysis device that provides an increased saturation range by comprising an increased number of probes as compared to other high-density analysis devices in the art that are in the sub-micron levels.

Alternatively, or in combination with other embodiments, the porous or non-porous material comprises a synthetic polymer, such as polyhydrocarbons, polyesters, nylon, and polycarbonates, or a bio-molecular aggregate including, such as, polysorbates, polylysine, DNA films, and carbohydrate-crosslinked gels.

There is also a method of detecting a bio-molecule comprising the steps of attaching an activatable material to a substrate, such that the activatable material and the substrate are in direct contact, to form a surface material, immobilizing a porous or nonporous material onto the surface material, reacting the porous or non-porous material with a probe to provide an element, repeating the step of reacting the porous or non-porous material to provide a high-density analysis device comprising a plurality of elements having a characteristic probe, applying a sample comprising a bio-molecule to the high-density analysis device, binding the bio-molecule to the probe, and detecting said binding.

A skilled artisan recognizes that each element is homogeneous for a probe, and the probe is characteristic for the element. In a specific embodiment, the step of reacting the porous or non-porous material with the probe is performed before the step of immobilizing the material. This means that the porous or non-porous material that is immobilized onto the surface material, which is in a solid state or in a near-liquid state, is pre-coated with a probe. The step of immobilization is then performed to provide an element, wherein the element is defined by a characteristic probe.

In some cases, the activatable material is characterized by having an activating temperature of less than 150° C. and comprises an adhesive, wherein the adhesive is thermally-cured.

The step of immobilizing may be achieved, for example, by trapping the porous material onto the surface material, wherein the surface material is in direct contact with at least one pore of the porous material. Another specific embodiment provides that the porous or non-porous material comprises an organism having a dimension in the range of about 1 micrometer to about 100 millimeters. Alternatively, the porous or non-porous material is characterized by having a diameter of at least about 1 micrometer.

In other specific applications of the method, the probe comprises a nucleic acid sequence, wherein the nucleic acid sequence is complementary to a nucleic acid sequence of the bio-molecule, or comprises a polypeptide having a complementary molecule, wherein the bio-molecule comprises the complementary molecule.

Further, the probe is an antibody having a complementary antigen, wherein the bio-molecule is the antigen. The antibody of the present invention includes fragments of the antibody that retain the functional activity of complementing the antigen. In other words, if an F_{ab} fragment or an epitope comprised therein of an antibody selectively binds (i.e., hybridizes to) the antigen, then the fragment is contemplated to be within the scope of the term “antibody”.

In another specific embodiment, the probe is a polypeptide having a biological activity, wherein the bio-molecule alters the biological activity of the polypeptide. A skilled artisan recognizes that there are methods in the art that allow functional assays of a polypeptide based on biological activity by, for example, catalyzing, increasing or enhancing the biological activity of a polypeptide, such as an enzyme. In the converse, a bio-molecule may decrease, inhibit, prevent and/or quench the biological activity of a polypeptide, such as an enzyme. In these cases, the biological activity is altered and the altered activity is detectable.

Alternatively, or in combination with other embodiments, the probe is labeled with a compound that fluoresces. Compounds that fluoresce are well known in the art and include, rhodamine and its derivatives, fluorescein and its derivatives, BODIPY® dyes, and Texas-Red. It is understood that the labeled probe is labeled to facilitate detection of binding to the target or bio-molecule. For example, a probe labeled with a fluorescent compound is analyzed/detected by fluorescence spectroscopy, which is a light dependent technique. However, other detection means are contemplated including refractive index, which is a density dependent technique, pH dependent techniques and multi-spectral imaging techniques such as chemiluminescence.

The present invention is also directed to a method of manufacturing a high-density bio-reactor)comprising the steps of attaching an activatable material to a substrate, such that the activatable material and the substrate are in direct contact, to form a surface material, immobilizing a porous or non-porous material onto the surface material, reacting the porous or non-porous material with a probe to generate an element, repeating the step of reacting the porous or non-porous material to provide the high-density bio-reactor comprising a plurality of elements having a characteristic probe, applying a sample comprising a target to the high-density bio-reactor, wherein an interaction between the target and the probe produces a detectable product, and detecting said product.

In one example of the method, the step of reacting the porous or nonporous material with the probe is performed before the step of immobilizing the porous or non-porous material. In another example, the activatable material is characterized by having an activating temperature of less than 150° C. Additionally, the activatable material may comprise an adhesive, wherein the adhesive is thermally-cured or is photocured.

It is preferred that the step of immobilizing is achieved by trapping the porous material onto the surface material, wherein the surface material is in direct contact with at least one pore of the porous material, and the porous material is at least 1 micrometer in diameter to provide a high target saturation level.

In other specific embodiments, the target is a polypeptide of an amino acid sequence of an enzyme, and, further, the probe is an organic molecule that serves as a substrate or reactant for the enzyme. The interaction of the probe with the target involves a selective interaction of the probe to the target and includes an enzyme-substrate binding such as a physical complementary mechanism (i.e., lock and key) or a geometrical complementary mechanism (i.e., allosterism), and a hybridization of complementary sequences, as for example if the organic molecule comprises a oligonucleotide tag. The interaction is performed under conditions that produce the detectable product, such as an enzymatic product. The product is detected by methods known in the art, such as mass spectrometry, chromatography, nuclear magnetic resonance spectroscopy and multi-spectral imaging techniques.

There is also a method of detecting expression of a polypeptide in a sample comprising the steps of attaching an activatable material to a substrate, such that the activatable material and the substrate are in direct contact, to form a surface material, immobilizing a porous or non-porous material onto the surface material, reacting a unit of the material with a probe, wherein the unit is homogenous for the probe, repeating the step of reacting a unit of the porous or non-porous material to provide to a high density biosensor comprising a plurality of units, wherein each unit is homogeneous for a probe, applying the sample comprising the polypeptide to the high-density bio-sensor, binding the polypeptide to the probe of at least one unit, and detecting said binding, wherein said detection indicates expression of said polypeptide in said sample.

The step of reacting the porous or non-porous material with the probe may be performed, for example, before the step of immobilizing the material.

In other specific embodiments, the activatable material is characterized by having an activating temperature of less than 150° C., and comprises an adhesive, wherein the adhesive is thermally-cured.

In a specific example, the step of immobilizing is achieved by trapping the porous material onto the surface material, wherein the surface material is in direct contact with at least one pore of the porous material, and the porous material is characterized by a having a diameter of at least 1 micrometer.

The probe, in some cases, may comprise a complementary DNA, an oligonucleotide, a chromosome, a PCR product or a gene fragment; alternatively, the probe comprises a polypeptide, an antibody, an antibody fragment having characteristic functional and/or structural activity or any combination thereof.

The present invention is also directed to immobilizing a porous or nonporous material on a substrate, and coating the porous or non-porous material with a probe of interest to manufacture a high-density microarray, wherein the high density refers to both the number of molecules per area of the porous or non-porous material and the increase in the surface area due to the three-dimensional nature of the porous or nonporous surface. This dramatic increase in the surface area over the prior art allows for an increase in the capture number of targets, hence increasing the dynamic range of the target binding. As described herein, the immobilization is a physical, non-covalent interaction between an surface material comprising an activatable material atop a substrate and a porous or non-porous material. It is preferred that the porous or non-porous material has a diameter of at least 1 micrometer (1 μm) to provide the increase in capture number of targets and increase in target saturation levels. Because of the relatively large size of the porous or non-porous material, the probe is bound to the outer and inner surfaces. Although immobilization renders some of the probes inert, the overall increase in available molecules per area compensates for the loss due to immobilization by infusing at least one pore with the surface material.

The present invention described herein provides a method for making microarrays comprising uniform elements that are characterized by a probe. In specific embodiments, the probe density on the array is varied by increasing the dimensions of the shape. Because molecules are not spotted as described in the prior art, the bead immobilization allows for improved uniformity and reproducibility.

In specific applications, the present invention is directed to manufacturing a high-density molecular analysis device comprising a porous or nonporous material coated with a probe such as a nucleic acid, a protein or an antibody. The methods of the present invention are well suited for such applications involving biomolecules because there is no direct dispensing of the probe onto the surface of the support, thus, there is little or no risk of cross contamination.

The present invention provides a simple method of manufacturing a microarray comprising attaching an activatable material to a substrate, such that the activatable material and the substrate are in direct contact, to form a surface material, immobilizing a porous or non-porous material onto the surface material, and reacting the porous or non-porous material with a probe to provide an element, wherein the microarray comprises a plurality of elements. The high density microarray results from the three-dimensional property of the porous or non-porous material and the size of the porous or non-porous material, which is preferably at least 1 micrometer in diameter. In specific embodiments, the porous or non-porous material is a glass having a three-dimensional shape, such as a sphere, and its surface is coated with a biomolecule by methods known in the art. The biomolecule is, by way of example, a nucleic acid that is bound to the porous or non-porous glass bead by a covalent or a non-covalent means (i.e., ionic interaction). This binding or reacting step is performed separately prior to the immobilization of the bead onto the support coated with, by way of example, an adhesive, wherein the adhesive is photo-curable such as an epoxide or thermally-curable such as a sol-gel glass. Alternatively, the binding or reacting step occur after the porous or non-porous material is immobilized onto the surface material. The resulting method is simple, decreases the potential for contamination from free floating probe molecules that plague the spotting methods of conventional microarray manufacturing and provides an increased target saturation level because of the size of the porous or non-porous material preferably being at least 1 micrometer, and the three-dimensional nature of the spatially addressable elements of the array.

Methods of binding a biomolecule such as a nucleic acid, a polypeptide, an antibody and/or antigen, a cell, an organelle include covalent and non-covalent methodologies. For example, dendrimer-reagent preparations having different analyte specificities have been immobilized on a solid phase as described in U.S. Pat. No. 6,121,056, solid supports comprising polymeric materials such as ethylene acrylic acid or ethylene methacrylic acid copolymers and activated polypropylene have been described for immobilizing biopolymers in U.S. Pat. No. 6,146,833. Binding is contemplated to involve a photo-cured adhesive or a photo-reactive molecule such as those described in U.S. Pat. No. 6,254,634, which describes coating compositions used to photoimmobilize a biomolecule; U.S. Pat. No. 6,278,018, which teaches a photoreactive molecule; U.S. Pat. No. 6,156,345 which teaches crosslinkable macromer systems; and U.S. Pat. No. 6,121,027 which teaches polybifunctional reagents. In a preferred embodiment of the present invention, a probe is reacted or bound to the porous or non-porous material via noncovalent interactions, and more preferably through ionic interactions.

The inventive device overcomes problems associated with conventional microarrays, which are typically two-dimensional and are directed to non-porous materials. The invention provides methods of manufacturing microarrays having a higher probe density because of the increased in the surface area over conventional microarrays. The porous or non-porous material which is a specific shape, for example, a rod uniform in diameter; hence each element of the array is made uniform, thereby allowing for the quantitation comparison of a microarray element within each and between other microarrays. Alternatively, the present invention also allows for the assay of several different kinds of probes by manufacturing an element having a characteristic probe. For example, a nucleic acid and protein are analyzed by two different elements of the same microarray. In the event that cross contamination is a major concern, the present invention contemplates that the immobilization of the probe onto the porous or non-porous material is performed prior to the immobilization of the porous or non-porous material to the substrate.

The invention further provides methods to analyze binding of a target using inherent chemical, physical and/or functional properties that are complementary to a chemical, physical and/or functional property of the probe. One such example is binding of an oligonucleotide probe to its complementary target, which is an oligonucleotide having a complementary nucleic acid sequence to the probe. Preferred nucleic acids for use in the subject invention are derivatized to contain at least one reactive moiety. Preferably the reactive moiety is at the 3′ or 5′ end. Alternatively, a nucleic acid is synthesized with a modified base. In addition, modification of the sugar moiety of a nucleotide at positions other than the 3′ and 5′ position is possible through conventional methods. Also, nucleic acid bases are modified, e.g., by using N7- or N9-deazapurine nucleosides or by modification of C-5 of dT with a linker arm, e.g., as described in F. Eckstein, ed., “Oligonucleotides and Analogues: A Practical Approach,” IRL Press (1991). Alternatively, backbone-modified nucleic acids (e.g., phosphoroamidate DNA) are used so that a reactive group is attached to the nitrogen center provided by the modified phosphate backbone. Alternatively, nucleic acids or any probe further comprises a linker, through which the nucleic acid is bound to the porous or non-porous material. Alternatively, the electrostatic and electronic properties of the probe are exploited to attach to the porous or non-porous material through a non-covalent interaction.

Preferably, modification of a nucleic acid, e.g., as described above, does not substantially impair the ability of the nucleic acid or nucleic acid sequence to hybridize to its complement. Thus, any modification preferably avoids substantially modifying the functionality of the nucleic acid which are responsible for Watson-Crick base pairing. The nucleic acid is modified such that a non-terminal reactive group is present, and the nucleic acid, when immobilized to the support, is capable of self-complementary base pairing to form a “hairpin” structure having a duplex region.

The method also includes a means of creating devices to detect, diagnose, identify and quantitate a molecule and/or a molecular interaction. Molecules contemplated include, for example, compounds of a biological, organic, synthetic, inorganic, metallic, and polymeric nature.

Some methods provided by this invention involve immobilization of the porous or non-porous material coated with a (probe) biomolecule (any and all molecules in the body of an organism either naturally or taken in any form from outside sources), or a synthetic molecule (any and all molecules artificially manufactured), or an organism, or cell, or organelle to a surface that is coated with a surface coating (molecular layer) that is activated by a variety of means to trap the porous or non-porous material on the surface coating. A target with a label is allowed to bind to the probe on the surface of the immobilized porous or non-porous material. A signal is detected and quantitated by using an imager of any origin to estimate the amount of the target.

### EXAMPLES

The following are illustrative, non-limiting examples of the some of the embodiments of the present invention. The following examples are included to demonstrate preferred embodiments of the invention. It should be appreciated by those skilled in the art that the techniques disclosed in the examples which follow represent techniques discovered by the inventor to function well in the practice of the invention, and thus is considered to constitute preferred modes for its practice. However, those of skill in the art should, in light of the present disclosure, appreciate that many changes made in the specific embodiments which are disclosed and maintain a like or similar result without departing from the concept, spirit and scope of the invention. More specifically, it is apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Methods—Preparation of the Surface

A support substrate is coated with layer of an activatable material. In specific embodiments, the activatable material is a glue, resin, wax, a sol-gel glass or a ceramic. The activatable material is applied to the substrate to at a known thickness to form a surface material. The thickness of the surface material does not exceed the diameter or the height of the porous or non-porous material that is to be immobilized as detailed below, by any of the means described earlier:

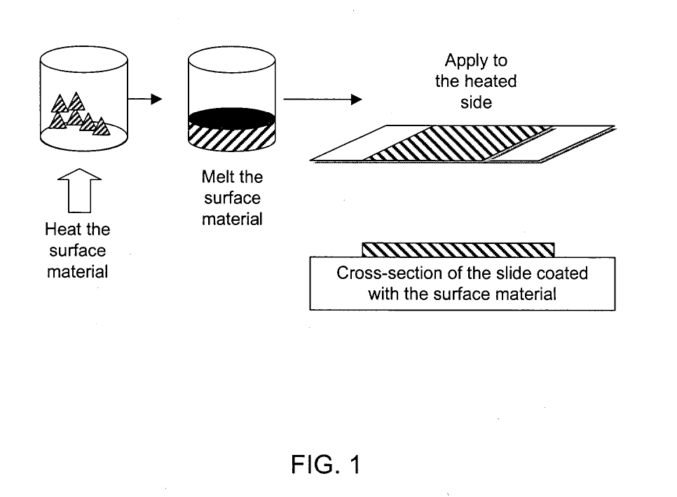

In a thermal deposition, the activatable material is heated to a temperature above the melting temperature and applied by any standard means to a heated or cooled slide. A surface material layer of a known thickness is made by controlling the amount of activatable material applied. The slide is cooled, allowing the surface to harden, forming a layer or crust on top as shown in FIG. 1.

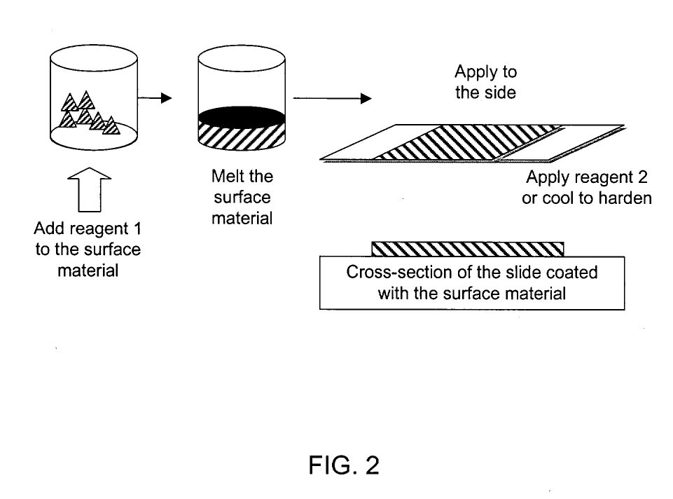

In a chemical deposition, the activatable material is treated with a first reagent that effects a transition to a soluble fluidic physical state phase and then is applied to a solid substrate such as a glass slide. The resulting layer is the surface material and is of known thickness, which is made by controlling the amount of fluidic activatable material applied. The surface material is attached by allowing it to dry in ambient conditions or, optionally, is treated with a second reagent to facilitate drying. Such reagents include organic solvents that are volatile and evaporate at ambient conditions more readily than aqueous solutions. FIG. 2 illustrates the coating of a substrate with a sol-gel activatable surface by chemical means.

### Example 2

### Methods—Immobilization of the Porous or Non-Porous Material

Porous or non-porous material with the molecule of interest attached to it is dispensed using microarray manufacturing methods, such as and not limited to contact, and non-contact methods on to the slide coated with the thermally or chemically solubulizable surface. The slide is heated in an oven for the thermal modification or treated with a reagent for chemical modification. The surface melts in both the cases in 2 above and the surface coating penetrates the porous or non-porous material. Hardening of the surface coating is accomplished either thermally or chemically. Once the surface hardens, the porous or non-porous material is embedded in the surface.

In an alternative specific embodiment, the porous or non-porous material is immobilized onto the surface material using a light or an electromagnetic wave to melt, bond, or physically adsorb the porous or non-porous material to the surface of the slide or substrate. In another specific embodiment, the porous or non-porous material is immobilized onto the surface material using a pressure, or force to embed the porous or non-porous material. Alternatively, a sound wave, a laser, a magnetic force, or an electrical force embeds and immobilizes the porous or non-porous material into the surface material.

Any glass, metal, ceramic, synthetic, organic or biological assembly is deemed to be a porous or non-porous material. The dimensions of the porous or nonporous materials can range from nanometers to millimeters. The microarrays produced have dimensions in the range of between about 1 nanometer to about 1 millimeter and have a probe density of about 1 to about 10,000/cm².

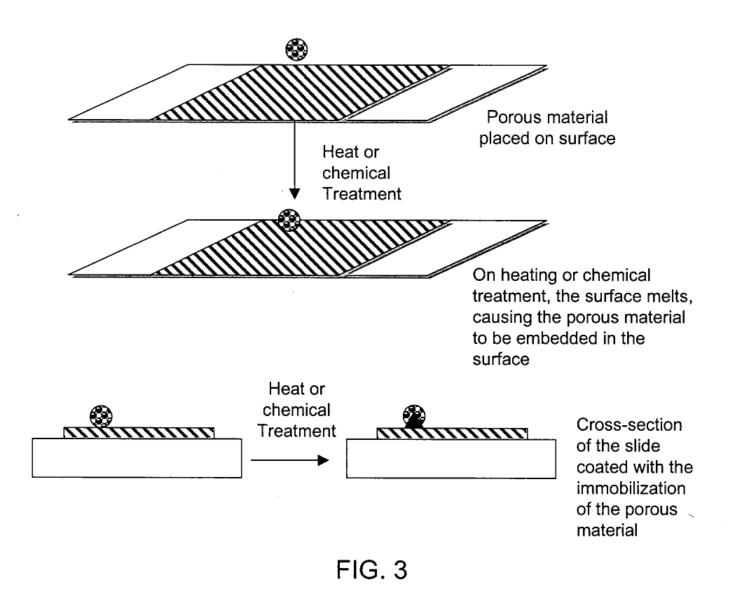

FIG. 3 illustrates the immobilization of a porous or non-porous material on the surface material. FIGS. 4A and 4B depict a cross-section of a porous or non-porous sphere A) before immobilization, and B) after immobilization to the surface material.

### Example 3

### Method of Manufacturing a Diagnostic Analysis Device

In an embodiment of the present invention is a method to manufacture a diagnostic device that is prepared to permit identification of a target based on functional properties. In a specific embodiment the diagnostic device identifies the nature of the target by an affinity of the target to its specific and complementary probe. For example, a nucleic acid sequence encoding a gene of an organism is bound to the porous or nonporous material through a covalent bond to produce a coated porous or non-porous material. The coated porous or non-porous material is then immobilized using the methods described herein to a surface material comprising a substrate and an activatable material that is direct contact with the substrate. A target that is complementary to probe is identified from a sample comprising a collection of targets by, for example, hybridization to the probe. In a specific embodiment, the target is a diagnostic for the presence of, for example, a pathogen in the sample. In another specific embodiment, the diagnostic device is used for forensic purposes or any other utility requiring identification of a target at the genomic level.

### Example 4

### Method of Manufacturing a High-Throughput, High-Density Bio-Sensor

The invention provides a means of generating a device to simultaneously investigate a genome or parts thereof of an organism. Depending on the dimensions of the porous or non-porous material, i.e. nano- to micro- to millimeter scale, the device manufactured using this invention is capable of analyzing millions of targets to a single probe.

In this sense, the present invention provides a means of manufacturing a microarray. A microarray includes spatially resolved elements of immobilized porous or non-porous material, with each element comprising a characteristic probe. The microarray comprises a collection of the elements to which a collection of targets from one or more samples is analyzed. A binding between a probe and a complementary target is identified and quantitated. Multiple assays are performed in parallel because the overall heterogeneous and three-dimensional nature of the microarray, wherein each element is homogeneous for a characteristic probe.

Further, in a specific embodiment, the probe is a live cell adsorbed to the porous or non-porous material. Applying a sample comprising a solution of a potential pharmaceutical to the high density and three dimensional device comprising elements having a characteristic probe and increased capture number for targets allows the measurement of drug response.

### Example 5

### Method of Manufacturing a High-Throughput, High-Density Analysis Device

The invention provides a means of generating a device for the simultaneous analysis of more than one gene or more than one protein expression profile using a probe comprising a cDNA, an oligonucleotide, a chromosomes, a PCR product, a gene fragment, wherein the gene fragment is a nucleic acid sequence that encodes for a gene that lacks an entire coding sequence, a polypeptide, an antibody, wherein the antibody includes monoclonal, polyclonal, chimerical, humanized, and artificial antibodies or fragments thereof, immobilized onto the porous or non-porous material. Simply, a unit of the porous or non-porous material is coated with a specific probe and immobilized on to a surface material such that the device comprises a plurality of units, wherein the probe of any one unit is different from the probe of any other unit. The porous or non-porous material coated with the specific probe is dispensed on to the surface using microarray manufacturing methods, such as and not limited to contact, and non-contact methods. Samples that are extracted from an organism, an organ, a tissue, a cell, or an organelle are allowed to selectively bind to the probe. The samples are allowed to interact with the probe for a time sufficient to detect levels of a target captured or bound to the probe as compared to the control sample. The gene and/or protein expression pattern is detected by, for example, imaging techniques that are known in the art such as chemiluminescence and autoradiography.

### Example 6

### Method of Manufacturing a High-Density Analysis Device for Identifying Genetic Alterations

The invention provides a means of generating a device to detect the difference (for example, fluorescence) between matched and mismatched probe-target complexes. This difference is used to identify mismatched or confirm matched probe-target complexes. In one specific embodiment, a mutation in a nucleic acid sequence encoding a gene or gene fragment obtained from a cell extract of an organism is identified as a single nucleotide polymorphism (SNP). A mismatched probe-target complex show a dampened, decreased or no signal as compared to matched probe-target complexes, thereby allowing the identification of an altered target to the a resolution of single nucleotide.

### Example 7

### Immobilization of the Porous or Non-Porous Beads on Thermally Modifiable Surfaces

The following procedure provides an illustrative but non-limiting example for the immobilization of beads described in the invention onto thermally activatable surface (paraffin) by non-covalent means. The surface of the device was made by melting a high-melting temperature (80° C.) paraffin (purified paraffin) and casting a 1 mm thick gel on a glass slide. The paraffin was allowed to solidify by cooling it to room temperature.

1. A sample of approximately 50 μ diameter porous or non-porous beads was dispersed in water. An aliquot of the sample was spotted on the paraffin surface.

2. The bead spotted paraffin surface was heated in the oven for a minute to allow for the beads to sink into the melted paraffin.

3. The slide was allowed to cool to room temperature and washed to ensure the trapping of the bead in the paraffin surface.

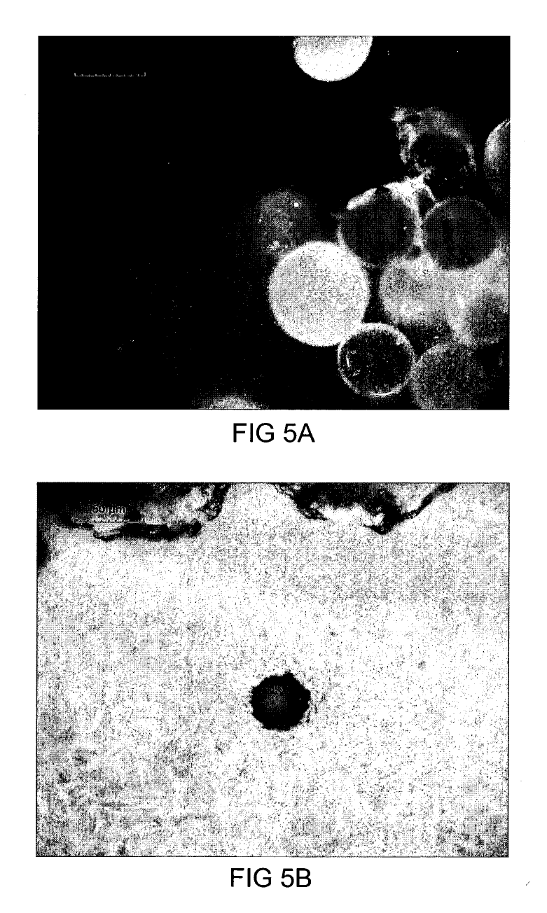

FIG. 5A shows beads before imbedding into the paraffin surface. A test spot is shown in the inset. FIG. 5B shows beads imbedded into the paraffin surface by thermal activation. A test spot is shown in the inset.

### Example 8

### Genotyping Using a Bead Microarray on Thermally Activatable Surfaces

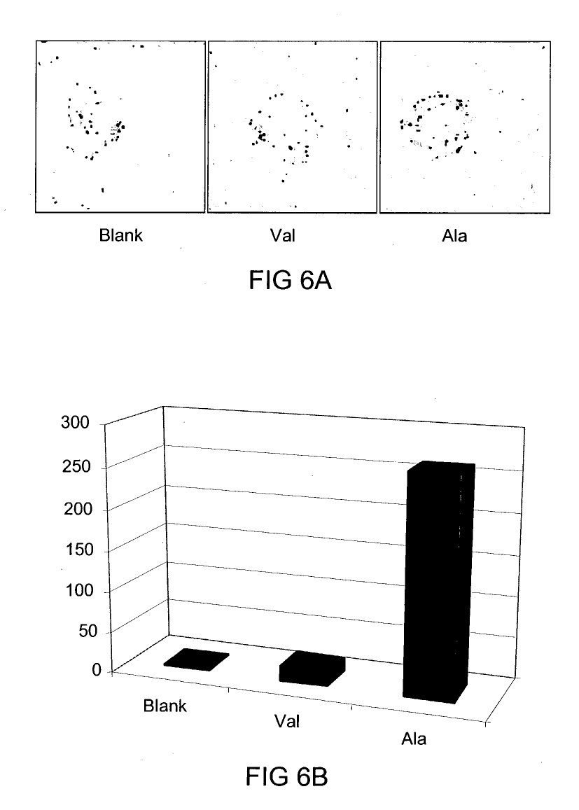

The bead microarrays were prepared as explained above, except that synthesized oligonucleotides corresponding to the two alleles that differ by a single nucleotide polymorphism (a single base change or mutation) in the gene for Manganese Superoxide Dismutase (MnSOD) were attached covalently to the bead (labeled Ala, which is a sequence of oligonucleotide with a central GCT). On a second set of beads, another oligonucleotide for different mutation (labeled Val where the central trio of bases was GTT) at the same location in the MNSOD gene was attached. Each of the bead sets was dispensed on a specific spatial location on the surface of the paraffin slide and immobilized as explained above. Targets corresponding from a human sample (breast tissue) were taken and amplified using PCR primers designed for this purpose (Forward PCR primer -biotin- 5′CCCAGCCTGCGTAGA3′ and Reverse PCR primer biotin-5′CGTCGTAGGGCAGGTCG3′). The amplified product was allowed to hybridize for 4 hours at room temperature in a standard hybridization buffer (5× SSC, 0.01M EDTA (pH 8.0), 5× Denhardt's solution, 0.5% (w/v) sodium dodecyl sulfate (SDS)). Following the hybridization, the microarrays were washed in washing buffer (0.1× SSC, 0.5% SDS) for 30 minutes. 5 μl solution containing 1 μg/ml (in phosphate buffered saline, pH 7.0) fluorescent dye Phycoerythrin conjugated to streptavidin was added to the array and incubated for 30 minutes in the dark. The arrays were washed in phosphate buffered saline and imaged using a microarray imager with a CCD camera at 5μ resolution. The intensities were quantified by integrating the pixels in each of the beads. A random oligonucelotide sequence was used as a negative control, labeled as blank. The mean fluorescent intensity was 2.0 for the blank, 17.5 for the Val beads and 263.0 for the Ala bead, indicating that sample contained the Ala mutation. FIG. 6A shows the fluorescence intensity of the blank, and the valine and alanine mutations immobilized onto beads, demonstrating the specificity of the method. FIG. 6B shows the relative fluorescence intensities in graphical form.

### References

All patents and publications mentioned in the specification are indicative of the levels of those skilled in the art to which the invention pertains. All patents and publications are herein incorporated by reference to the same extent as if each individual publication was specifically and individually indicated to be incorporated by reference herein.

### Patents

U.S. published application 2001/0039072

U.S. Pat. No. 5,900,481 to Lough et al.

U.S. Pat. No. 6,133,436 to Koster et al.

U.S. Pat. No. 5,837,196 to Pinkel et al.

U.S. Pat. No. 5,436,327 to Southern et al.

U.S. Pat. No. 6,139,831 to Shivashankar et al.

U.S. Pat. No. 5,412,087 to McGall et al.

U.S. Pat. No. 6,327,410 to Walt et al.

U.S. Pat. No. 5,807,522 to Brown et al.

U.S. Pat. No. 5,900,215

U.S. Pat. No. 6,121,056

U.S. Pat. No. 6,146,833

U.S. Pat. No. 6,254,634

U.S. Pat. No. 6,278,018

U.S. Pat. No. 6,156,345

U.S. Pat. No. 6,121,027

WO 90/03382

WO 93/22680

WO 95/15970

WO 00/61282

### Non-Patented Literature

Shi, L., “DNA Microarray (Genome Chip)—Monitoring the Genome on a Chip”, http://www.gene-chip.com.

Lehrach, et al., 1990

Pirrung, et al. (1992)

Fodor, et al. (1991)

Khrapko, et al. (1991)

Maniatas, et al., 1989—MOLECULAR CLONING, A LABORATORY MANUAL, Cold Spring Harbor Press (1989).

Merrifield, J., J. Am. Chem. Soc., 91:501 (1969).

Chaiken, I. M., CRC Crit. Rev. Biochem., 11:255 (1981).

Kaiser et al., Science, 243:187 91989).

Merrifield, B., Science, 232:342 (1986).

Kent, Ann. Rev. Biochem., 57:957 (1988).

Offord, R. E., Semisynthetic Proteins, Wiley Publishing (1980).

Foder et al., Science, 251:767-773 (1991).

Cho et al., Science, 261:1303-1305 (1993).

Bachmann et al., “Advances in Light Curing Adhesives”.

F. Eckstein, ed., “Oligonucleotides and Analogues: A Practical Approach,” IRL Press (1991).

Advances in Biosensors, A. P. F. Turner, Ed. JAI Press, London, (1991).

Updike and Hicks, Nature, 214: 986 (1967).

Abdel-Latifet al., Anal. Lett., 21: 943 (111988).

Giaever, J Immunol., 110: 1424 (1973).

Sugao et al. Anal. Chem., 65: 363 (1993).

Rogers et al. Anal. Biochem., 182: 353 (1989).

One skilled in the art readily appreciates that the patent invention is well adapted to carry out the objectives and obtain the ends and advantages mentioned as well as those inherent therein. Materials, reactions, sequences, methods, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

What is claimed is:

1. A method of manufacturing a high-density molecular analysis device comprising
i) attaching an activatable material to a substrate, such that the activatable material and the substrate are in direct contact, to form a surface material;
ii) immobilizing a porous material onto the surface material;
iii) reacting the porous material with a probe to generate an element; and
iv) repeating step iii) to provide the high-density molecular analysis device comprising a plurality of elements, wherein each element is homogeneous for the probe of that element.

2. The method of claim 1, wherein the step of reacting the porous material with the probe is performed before the step of immobilizing the porous material.

3. The method of claim 1, wherein the activatable material is characterized by having an activating temperature of less than 150° C.

4. The method of claim 1, wherein the activatable material is a polymer.

5. The method of claim 4, wherein said polymer is agarose.

6. The method of claim 5, wherein said probe is a cell.

7. The method of claim 1, wherein the activatable material is a sol-gel glass.

8. The method of claim 1, wherein the activatable material comprises an adhesive, wherein the adhesive is thermally-cured.

9. The method of claim 8, wherein the adhesive is an epoxide, an acrylic, a wax, a resin or an organic molecule that functions as a synthetic support.

10. The method of claim 1, wherein the activatable material comprises an adhesive, wherein the adhesive is photo-cured.

11. The method of claim 10, wherein the adhesive is an epoxide or an acrylic.

12. The method of claim 1, wherein said the step of immobilizing is achieved by trapping the porous material onto the surface material, wherein the surface material is in direct contact with at least one pore of the porous material.

13. The method of claim 1, wherein said porous material is characterized by having a diameter of at least about 1 micrometer.

14. The method of claim 1, wherein said porous material comprises a synthetic polymer.

15. The method of claim 1, wherein said porous material comprises a biomolecular aggregate.

16. A method of manufacturing a high-density molecular analysis device comprising
i) attaching an activatable material to a substrate, such that the activatable material and the substrate are in direct contact, to form a surface material;
ii) immobilizing a porous or non-porous material onto the surface material;
iii) reacting the porous or non-porous material with a probe to generate an element; and
iv) repeating step iii) to provide the high-density molecular analysis device comprising a plurality of elements, wherein each element is homogeneous for the probe of that element.

17. A method of detecting a bio-molecule comprising the steps of:
i) attaching an activatable material to a substrate, such that the activatable material and the substrate are in direct contact, to form a surface material;
ii) immobilizing a porous material onto the surface material;
iii) reacting the porous material with a probe to provide an element;
iv) repeating step iii) to provide a high-density analysis device comprising a plurality of elements having a characteristic probe;
v) applying a sample comprising a bio-molecule to the high-density analysis device;
vi) binding the bio-molecule to the probe; and
v) detecting said binding.

18. The method of claim 17, wherein the step of reacting the porous material with the probe is performed before the step of immobilizing the porous material.

19. The method of claim 17, wherein the activatable material is characterized by having an activating temperature of less than 150° C.

20. The method of claim 17, wherein the activatable material comprises an adhesive, wherein the adhesive is thermally-cured.

21. The method of claim 17, wherein said the step of immobilizing is achieved by trapping the porous material onto the surface material, wherein the surface material is in direct contact with at least one pore of the porous material.

22. The method of claim 17, wherein said porous material comprises an organism having a dimension in the range of about 1 micrometer to about 100 millimeters.

23. The method of claim 17, wherein said porous material is characterized by having a diameter of at least about 1 micrometer.

24. The method of claim 17, wherein said probe comprises a nucleic acid sequence, wherein the nucleic acid sequence is complementary to a nucleic acid sequence of the bio-molecule.

25. The method of claim 17, wherein said probe comprises a polypeptide having a complementary molecule, wherein the bio-molecule comprises the complementary molecule.

26. The method of claim 17, wherein said probe is an antibody having a complementary antigen, wherein the bio-molecule is the antigen.

27. The method of claim 17, wherein said probe is a polypeptide having a biological activity, wherein the bio-molecule alters the biological activity of the polypeptide.

28. The method of claim 17, wherein said probe is labeled with a compound that fluoresces.

29. The method of claim 28, wherein the step of detecting comprises fluorescence spectroscopy.

30. The method of claim 17, wherein said probe is a molecule that alters a biological activity of a polypeptide, wherein the bio-molecule comprises the polypeptide.

31. A method of detecting a bio-molecule comprising the steps of:
i) attaching an activatable material to a substrate, such that the activatable material and the substrate are in direct contact, to form a surface material;
ii) immobilizing a porous or non-porous material onto the surface material;
iii) reacting the porous or non-porous material with a probe to provide an element;
iv) repeating step iii) to provide a high-density analysis device comprising a plurality of elements having a characteristic probe;
v) applying a sample comprising a bio-molecule to the high-density analysis device;
vi) binding the bio-molecule to the probe; and
v) detecting said binding.

32. A method of manufacturing a high-density bio-reactor comprising the steps of:
i) attaching an activatable material to a substrate, such that the activatable material and the substrate are in direct contact, to form a surface material;
ii) immobilizing a porous material onto the surface material;
iii) reacting the porous material with a probe to generate an element;
iv) repeating step iii) to provide the high-density bio-reactor comprising a plurality of elements having a characteristic probe;
v) applying a sample comprising a target to the high-density bio-reactor, wherein an interaction between the target and the probe produces a detectable product; and
vi) detecting said product.

33. The method of claim 32, wherein the step of reacting the porous material with the probe is performed before the step of immobilizing the porous material.

34. The method of claim 32, wherein the activatable material is characterized by having an activating temperature of less than 150° C.

35. The method of claim 32, wherein the activatable material comprises an adhesive, wherein the adhesive is thermally-cured.

36. The method of claim 32, wherein said the step of immobilizing is achieved by trapping the porous material onto the surface material, wherein the surface material is in direct contact with at least one pore of the porous material.

37. The method of claim 32, wherein said target is a polypeptide of an amino acid sequence of an enzyme.

38. The method of claim 37, wherein said probe is an organic molecule that serves as a substrate for said enzyme.

39. A method of manufacturing a high-density bio-reactor comprising the steps of:
i) attaching an activatable material to a substrate, such that the activatable material and the substrate are in direct contact, to form a surface material;
ii) immobilizing a porous or non-porous material onto the surface material;
iii) reacting the porous or non-porous material with a probe to generate an element;
iv) repeating step iii) to provide the high-density bio-reactor comprising a plurality of elements having a characteristic probe;
v) applying a sample comprising a target to the high-density bio-reactor, wherein an interaction between the target and the probe produces a detectable product; and
vi) detecting said product.

40. A method of detecting expression of a polypeptide in a sample comprising the steps of:
i) attaching an activatable material to a substrate to form a surface material, such that the activatable material and the substrate are in direct contact;
ii) immobilizing a porous material onto the surface material;
iii) reacting a unit of the porous material with a probe, wherein the unit is homogenous for the probe;
iv) repeating step iii) to provide to a high density bio-sensor comprising a plurality of units, wherein each unit is homogeneous for a probe;
v) applying the sample comprising the polypeptide to the high-density bio-sensor;
vi) binding the polypeptide to the probe of at least one unit; and
vii) detecting said binding, wherein said detection indicates expression of said polypeptide in said sample.

41. The method of claim 40, wherein the step of reacting the porous material with the probe is performed before the step of immobilizing the porous material.

42. The method of claim 40, wherein the activatable material is characterized by having an activating temperature of less than 150° C.

43. The method of claim 40, wherein the activatable material comprises an adhesive, wherein the adhesive is thermally-cured.

44. The method of claim 40, wherein said the step of immobilizing is achieved by trapping the porous material onto the surface material, wherein the surface material is in direct contact with at least one pore of the porous material.

45. The method of claim 40, wherein said probe comprises a complementary DNA, an oligonucleotide, a chromosome, a PCR product or a gene fragment.

46. The method of claim 40, wherein said probe comprises a polypeptide.

47. The method of claim 40, wherein said probe comprises an antibody.

48. The method of claim 40, wherein said probe comprises a synthetic small molecule.

49. The method of claim 40, wherein said probe comprises a whole viral particle.

50. The method of claim 40, wherein said probe comprises a viral coat protein assembly.

51. The method of claim 40, wherein said probe comprises a cell.

52. The method of claim 40, wherein said probe comprises a whole viral particle.

53. The method of claim 40, wherein said probe comprises a viral coat protein assembly.

54. The method of claim 40, wherein said probe comprises a microorganism.

55. A method of detecting expression of a polypeptide in a sample comprising the steps of:
i) attaching an activatable material to a substrate to form a surface material, such that the activatable material and the substrate are in direct contact;
ii) immobilizing a porous or non-porous material onto the surface material;
iii) reacting a unit of the porous or non-porous material with a probe, wherein the unit is homogenous for the probe;
iv) repeating step iii) to provide to a high density bio-sensor comprising a plurality of units, wherein each unit is homogeneous for a probe;
v) applying the sample comprising the polypeptide to the high-density bio-sensor;
vi) binding the polypeptide to the probe of at least one unit; and
vii) detecting said binding, wherein said detection indicates expression of said polypeptide in said sample.

56. A method of manufacturing a high-density molecular analysis device comprising
i) attaching a molecule or cell to a substrate, such that the molecule or cell and the substrate are in direct contact, to form a surface material;
ii) immobilizing a porous material onto the surface material by thermal modification;
iii) reacting the porous material with a probe to generate an element; and
iv) repeating step iii) to provide the high-density molecular analysis device comprising a plurality of elements, wherein each element is homogeneous for the probe of that element.

57. A method of manufacturing a high-density molecular analysis device comprising
i) attaching a molecule or cell to a substrate, such that the molecule or cell and the substrate are in direct contact, to form a surface material;
ii) immobilizing a porous or non-porous material onto the surface material by thermal modification;
iii) reacting the porous or non-porous material with a probe to generate an element; and
iv) repeating step iii) to provide the high-density molecular analysis device comprising a plurality of elements, wherein each element is homogeneous for the probe of that element.
